(19) United States
(12) Patent Application Publication
(10) Pub. No.: US 2015/0374257 A1
(43) Pub. Date: Dec. 31, 2015

(54) Measurement method for detecting vital parameters in a human or animal body, and measuring apparatus

(71) Applicant: LifeTAix GmbH, Aachen (DE)

(72) Inventors: Daniel TEICHMANN, Aachen (DE); Steffen LEONHARDT, Aachen (DE); Marian WALTER, Aachen (DE); Jerome FOUSSIER, Aachen (DE); Tobias WARTZEK, Aachen (DE)

(73) Assignee: LIFETAIX GMBH, Aachen (DE)

(21) Appl. No.: 14/670,100

(22) Filed: Mar. 26, 2015

(30) Foreign Application Priority Data
Mar. 28, 2014 (DE) 102014104465.7

(51) Int. Cl. A61B 5/05; A61B 5/00; A61B 5/024; A61B 5/11; A61B 5/0205; A61B 5/08

(52) U.S. Cl. CPC A61B 5/05; A61B 5/02055; A61B 5/08; A61B 5/02416; A61B 5/11; A61B 5/6891; A61B 5/6893; A61B 5/725

(57) ABSTRACT

A measurement method (1) for detecting vital parameters in a human or animal body, in which, in a detection step (3), a magnetic induction sensor detects an induction measurement sequence which is dependent on a time-varying change in at least one vital parameter, wherein in the detection step (3), a secondary sensor unit simultaneously detects a secondary measurement sequence, the secondary measurement sequence being dependent on an influential variable signal sequence that influences the induction measurement sequence, and in that in a subsequent combination step (4), at least one vital parameter measurement sequence for a vital parameter detected from the induction measurement sequence is calculated from the induction measurement sequence and the secondary measurement sequence using a predefined combination function, so that the detection accuracy of the vital parameter represented in the vital parameter measurement sequence and the induction measurement sequence is improved by combining the induction measurement sequence with the secondary measurement sequence to form the vital parameter measurement sequence. The invention also relates to a measuring apparatus for carrying out the method according to the invention.

The invention relates to a measurement method for detecting vital parameters in a human or animal body, in which, in a detection step, a magnetic induction sensor is used to obtain a sequence of induction measurements which is dependent on a time-varying change in at least one vital parameter.

Various methods for monitoring vital parameters are known in the prior art. For example, EP 2 777 491 A1 describes a measurement method for detecting vital parameters, in which a human or animal is irradiated with broadband electromagnetic radiation, and the vital parameters are determined based on the spectrum of the radiation that is reflected.

Document DE 10 2011 110 486 A1 describes a method and a device for monitoring the vital parameters of a driver of a vehicle in which optical images of the driver are analyzed. A further device for monitoring the vital parameters of a driver of a vehicle is described in DE 10 2012 002 037 A1. In the method described therein, electrocardiogram signals are captured and analyzed by a plurality of capacitive sensors installed in the driver's seat.

Document DE 10 2011 112 226 A1 describes a device for monitoring vital parameters which combines an optical monitoring of movements and pulse rate with a capacitive electric field measurement.

Measurement methods that use magnetic induction sensors are likewise already known and in use for the contact-free detection of vital parameters in a human or animal body. For example, tomographic images captured by a plurality of magnetic induction sensors arranged in a circle and monitoring vital parameters such as respiration and heart action are known.

To detect a vital parameter, the magnetic induction sensor is positioned on the body, and a sequence of induction measurements that reflect a time-varying change in heart action, for example, is recorded. The detection of vital parameters by means of magnetic induction sensors makes use of the conductive properties of bodily fluids. The signal-to-noise ratio of the obtained induction measurement sequence is substantially dependent on the distance of the magnetic induction sensor from the measurement point. Since there is a strong dependency relationship between the position of the magnetic induction sensor and both the strength of the measured signal and the signal-to-noise ratio, even slight body movements impact the measurement result. For example, during the detection of heart action by means of the magnetic induction sensor, respiratory movements of the lungs and micromovements of the body represent interfering components in the detection of heart action. However, since the nature of the interfering components in the induction measurement sequence is unknown, and since the interfering components cannot be distinguished from one another, the separation of the interfering components from the induction measurement sequence and the detection of vital parameters free from interfering components are made substantially more difficult.

It is therefore considered an object of the present invention to design the measurement method for detecting vital parameters in a human or animal body such that interfering components can be separated from induction measurement sequences obtained via the magnetic induction sensor, and such that vital parameters can thereby be detected by means of the magnetic induction sensor with a smaller interfering component.

This object is attained according to the invention in that, in the detection step, a magnetic induction sensor detects an induction measurement sequence, while at the same time, a secondary sensor unit detects a secondary measurement sequence, the secondary measurement sequence being dependent on an influential variable signal sequence that influences the induction measurement sequence, and in that in a subsequent combination step, at least one vital parameter measurement sequence for a vital parameter detected from the induction measurement sequence is calculated from the induction measurement sequence and the secondary measurement sequence using a predefined combination function, so that the detection accuracy of the vital parameter represented in the vital parameter measurement sequence and the induction measurement sequence is improved by combining the induction measurement sequence with the secondary measurement sequence to form the vital parameter measurement sequence.

In this manner, for example, the heart action of a human or an animal can be represented on a time-varying basis in the induction measurement sequence detected by the magnetic induction sensor, and movements of the body caused by respiration can be detected in the secondary measurement sequence. Subsequently, according to the invention, based on the detected secondary measurement sequence and the combination function, which is predefined by a user or measured, for example, the influential variable component of the induction measurement sequence can be diminished, allowing the heart action to be detected and interpreted without the interfering component that is generated by respiratory movement. The secondary sensor unit in this case can comprise one or more sensors that employ similar or different measuring principles. According to the invention, the secondary sensor unit can be positioned close to the magnetic induction sensor so as to detect movements of the body occurring at the measuring point of the magnetic induction sensor with the greatest possible accuracy.

A measurement method of this design allows the influential variable component to be at least partly offset and allows the vital parameters in the vital parameter measurement sequence to be detected more accurately. This is accomplished by separating out the influential variable components that are produced by movements of the body in the induction measurement sequence obtained by the magnetic induction sensor.

According to the invention, it is advantageously provided that prior to the detection step the combination function is established in a calibration step. The combination function can be established only once to initialize the measurement method to be used for a measurement, and can then be applied in the combination step to offset the influential variable component in the induction measurement sequence. The combination function advantageously generates a functional correlation between the induction measurement sequence, the secondary measurement sequence and the vital parameter to be detected. Possible parameters for the combination function include the distance of the magnetic induction sensor from the body and the amplitude of a constant noise component. The initial establishment of the combination function allows each combination function to be adapted to the conditions predetermined by the respective measuring apparatus or by the respective environment.

It is advantageously provided according to the invention that in the calibration step, parameters of the combination function are established. For example, the combination function can be predefined by a user, or the parameters of the predefined combination function can be established based on a measurement by means of a suitable identification process. Parameters of the combination function may include, for example, the distance of the magnetic induction sensor from the body or the ambient temperature. These parameters may also be supplemented by parameters that are predefined by the user and used in the combination function, for example. Establishing the parameters of the combination function prior to the detection step allows the combination function to be established based on the measuring conditions, thereby improving, in the combination step, the accuracy of detection of the vital parameter represented in the vital parameter measurement sequence.

According to one embodiment of the measurement method according to the invention, it is advantageously provided that, following the combination step, the vital parameter is determined from the vital parameter measurement sequence in an extraction step. For example, the vital parameter of heart rate can be extracted from the vital parameter measurement sequence obtained in the combination step, in which the influential variable has been offset and which represents the time-varying heart action. According to the invention, the extracted vital parameter can further be forwarded in the extraction step to an output unit, so that the vital parameter can be interpreted by medical personnel, for example.

In a particularly advantageous embodiment of the measurement method, it is provided according to the invention that the secondary measurement sequence is dependent on at least one additional time-varying vital parameter, and that the time-varying vital parameter detected from the secondary measurement sequence is the influential variable component of the induction measurement sequence. For example, heart action can be detected by the magnetic induction sensor in the induction measurement sequence, and respiratory action can be detected by the secondary sensor unit in the secondary measurement sequence. Respiratory movement influences the induction measurement sequence obtained by the magnetic induction sensor, and therefore represents the influential variable component of the induction measurement sequence.

According to the invention, it is provided that, in the combination step, the induction measurement sequence and the secondary measurement sequence are combined by means of a compensation method, in order to offset an undesirable influential variable component in the induction measurement sequence. The compensation method may involve, for example, subtracting individual, optionally correspondingly transformed measurement sequence values of the induction measurement sequence and of the secondary measurement sequence. However, it is also possible and provided according to the invention to use an adaptive filter to offset the influential variable component.

By applying the compensation method, the influential variable component in the induction measurement sequence, which variable component is detected by means of the secondary measurement sequence, can be offset in a simple manner. In detecting heart action, for example, it would be possible to offset the movement artefacts detected in the secondary measurement sequence by using an adaptive filter in the induction measurement sequence.

It is preferably provided that, in the combination step, the induction measurement sequence and the secondary measurement sequence are combined with one another by means of a complementary fusion method to form the vital parameter measurement sequence, to offset any detection errors. This allows the time dependency of a common vital parameter in both the secondary measurement sequence and the induction measurement sequence to be detected. Applying complementary fusion, for example addition, of the two measurement sequences, allows measuring periods during which vital parameters could not be detected or during which vital parameters could be detected only insufficiently by the magnetic induction sensor to be offset by the secondary measurement sequence.

According to the invention, it is provided that the time-varying vital parameter detected with the induction measurement sequence is a secondary influential variable component of the secondary measurement sequence, and that in the combination step, a mutual compensation method is used to offset the secondary influential variable component of the secondary measurement sequence based on the induction measurement sequence and a further predefined combination function in the secondary measurement sequence. According to the invention, in the combination step both the influential variable component of the secondary measurement sequence in the induction measurement sequence and the secondary influential variable component of the induction measurement sequence in the secondary measurement sequence can be diminished.

For example, in the induction measurement sequence, heart action can be detected by the magnetic induction sensor, and in the secondary measurement sequence, respiratory action can be detected by the secondary sensor unit. The heart action detected in the induction measurement sequence influences the measurement of respiratory action detected with the secondary measurement sequence, and conversely, the respiratory action detected with the secondary measurement sequence influences the measurement of heart action detected with the induction measurement sequence. In the combination step, the influential variable component of the respiratory movement represented in the secondary measurement sequence can then be diminished in the induction measurement sequence, and in the extraction step, the first vital parameter can be determined. In a similar manner, in the combination step, the secondary influential variable component of the heart action represented in the induction measurement sequence can be diminished in the secondary measurement sequence, and the second vital parameter can be determined in the extraction step.

Thus with the measurement method according to the invention, at least two vital parameters can be determined in the extraction step and simultaneously monitored.

According to the invention, it is provided that in the combination step, by combining the induction measurement sequence with the secondary measurement sequence using a source separation method based on a mathematical model which describes a correlation between the vital parameter represented in the induction measurement sequence and the secondary influential variable component represented in the secondary measurement sequence and/or the vital parameter represented in the secondary measurement sequence, at least one vital parameter measurement sequence is determined, with one vital parameter detected in the induction measurement sequence and/or in the secondary measurement sequence being represented in the vital parameter measurement sequence. If heart action is detected by the magnetic induction sensor in the induction measurement sequence and respiratory action is detected by the secondary sensor unit in the secondary measurement sequence, then, by applying the source separation method based on a mathematical model, the influential variable component of the respiratory action can be segregated from the heart action detected with the induction sequence, and the time dependency of the heart action detected with the induction measurement sequence can be represented in a first vital parameter measurement sequence, and the time dependency of the respiratory action detected from the secondary measurement sequence can be represented in a second vital parameter measurement sequence. According to the invention, the source separation method can be used for processing the induction measurement sequence and a plurality of secondary measurement sequences, and therefore the source separation method can be used to determine a plurality of vital parameter measurement sequences.

According to the invention, it is provided that the source separation method is implemented based on an independent component analysis, a Kalman filtering or a principal component analysis.

The invention also relates to a measuring apparatus used for detecting vital parameters in a human or animal body by means of the measurement method described above. According to the invention, it is provided that the measuring apparatus comprises a magnetic induction sensor, an analysis unit and a secondary sensor unit, with the analysis unit being connected to the magnetic induction sensor and to the secondary sensor unit so as to enable signal transmission. The induction measurement sequence obtained by the magnetic induction sensor and the secondary measurement sequence obtained by the secondary sensor unit can be forwarded via a signal-transmitting connection to the analysis unit, in which the influential variable component in the induction measurement sequence and, if applicable, the secondary influential variable component in the secondary measurement sequence can be offset by means of a mutual compensation method, for example. According to the invention, a source separation method, a complementary fusion method or a compensation method can also be carried out in the analysis unit. The magnetic induction sensor and the secondary sensor unit can be arranged in close proximity to one another, to allow the vital parameters to be detected at the same position.

Using the measuring apparatus comprising the magnetic induction sensor, the secondary sensor unit and the analysis unit, the measurement method as described above can be carried out such that the detection accuracy of the vital parameter represented in the vital parameter measurement sequence and the induction measurement sequence is improved by combining the induction measurement sequence with the secondary measurement sequence to form the vital parameter measurement sequence.

In a particularly advantageous embodiment of the measuring apparatus, it is provided according to the invention that the measuring apparatus comprises a storage unit, connected to the analysis unit so as to enable signal transmission, for storing the combination function and/or the induction measurement sequence and/or the secondary measurement sequence and/or the vital parameter measurement sequence. The storage unit allows the combination function, the induction measurement sequence, the vital parameter measurement sequence and/or the secondary measurement sequence to be stored and retrieved at any time for further calculation in the combination step or in the extraction step.

Advantageously, it is provided according to the invention that the secondary sensor unit comprises at least one secondary sensor for detecting the secondary measurement sequence. The secondary sensor can detect the secondary measurement sequence, which can then be used to offset the influential variable component in the induction measurement sequence. If the induction measurement sequence obtained by the magnetic induction sensor has no influential variable component, the induction measurement sequence will not be influenced during the subsequent calculation in the combination step.

It is provided that the secondary sensor uses a measuring principle different from that used by the magnetic induction sensor, in order, for example, to detect different vital parameters in the secondary measurement sequence and determine these in the extraction step, and in order to factor in different dependencies of different sensor types on environmental parameters and the like, thereby improving detection accuracy.

According to the invention, it is provided that the secondary sensor is an optical sensor. The optical sensor can be used, for example, for detecting pulse rate or body movement. The combination function or the parameters of the combination function can also be determined by means of the optical sensor and the magnetic induction sensor. Due to its smaller dimensions, the optical sensor can be positioned in close proximity to the magnetic induction sensor, so that the secondary measurement sequence and the induction measurement sequence can be detected at the same time and position.

Advantageously, it is provided according to the invention that the secondary sensor is an acceleration sensor. The acceleration sensor can be used, for example, for detecting body movements. Particularly if the measuring apparatus is positioned close to the body, the secondary measurement sequence can be detected accurately and simply.

It is provided that the secondary sensor is a sensor based on capacitance coupling. For example, the sensor can be a capacitive sensor for detecting heart action or some other vital parameter.

According to the invention, it is provided that the secondary sensor is a capacitive distance sensor. The capacitive distance sensor can be, for example, a radar sensor, an ultrasound sensor, or a capacitive sensor. The distance sensor can likewise detect body movements accurately and simply. It is also possible according to the invention to combine the secondary measurement sequences detected using different measuring principles with one another.

In a particularly advantageous embodiment of the measuring apparatus, it is provided according to the invention that

the measuring apparatus is integrated into an automobile seat, an examination chair or a hospital bed. Integration into an article of clothing is likewise provided according to the invention. By attaching the measuring apparatus close to the body, measurement errors that occur during detection of the induction measurement sequence and the secondary measurement sequence are reduced, enabling an accurate and error-free monitoring of vital parameters. Such positioning also enables an uninterrupted and easily implemented monitoring of vital parameters of automobile and truck drivers, patients being transported or monitored, and athletes, for example.

Additional advantageous embodiments of the measurement method according to the invention and the measuring apparatus according to the invention will be specified in greater detail in reference to embodiments represented in the set of drawings. The drawings show:

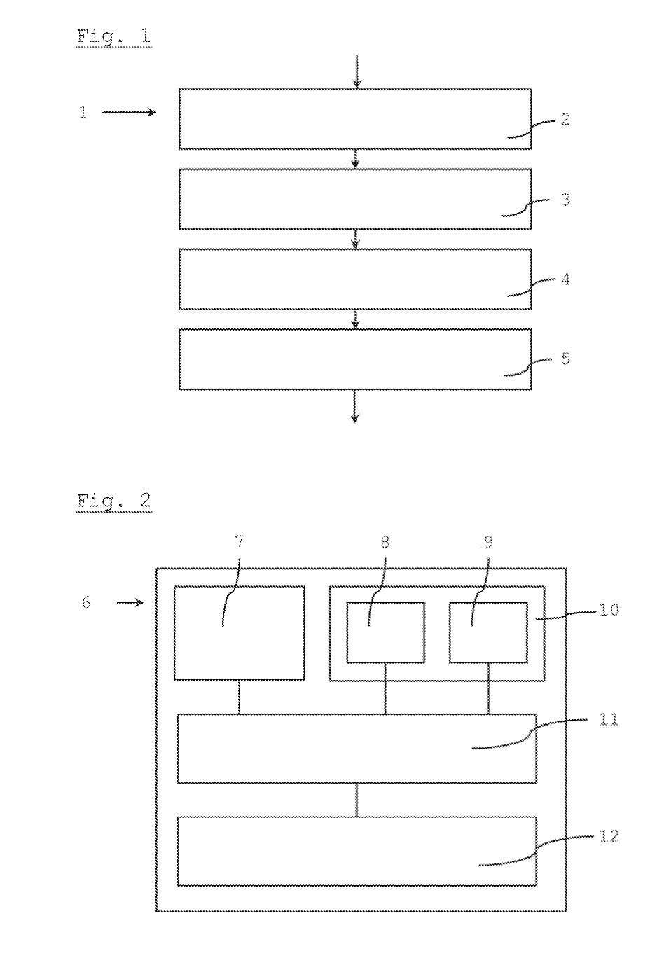

FIG. 1 a schematic flow chart of a measurement method according to the invention;

FIG. 2 a schematic view of a measuring apparatus according to the invention;

FIGS. 3 to 8 schematic views of possible arrangements of a magnetic induction sensor and secondary sensors;

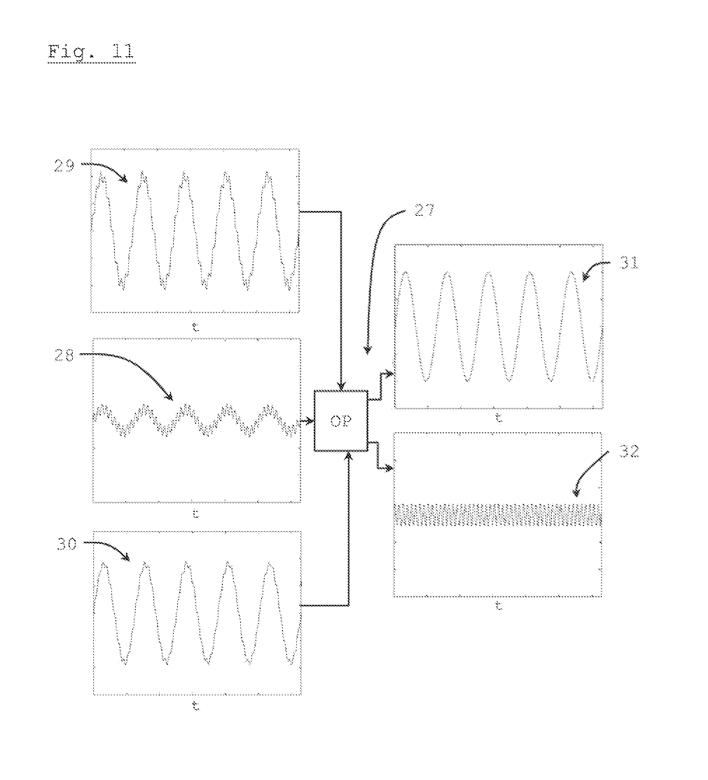

FIG. 9 a schematic representation of a combination step involving a compensation method;

FIG. 10 a schematic representation of a combination step involving a complementary fusion method;

FIG. 11 a schematic representation of a combination step involving a source separation method.

FIG. 1 shows a schematic flow chart of a measurement method 1 according to the invention. Measurement method 1 comprises a calibration step 2, a detection step 3, a combination step 4 and an extraction step 5.

In calibration step 2, a combination function can be established or, for example, predefined by a user. In detection step 3, a magnetic induction sensor detects an induction measurement sequence, which reflects a time-varying change in a vital parameter, and a secondary sensor detects a secondary measurement sequence, which is dependent on an influential variable signal sequence that influences the induction measurement sequence. In combination step 4, an influential variable component represented in the secondary measurement sequence is diminished in the induction measurement sequence by means of the combination function, and a vital parameter measurement sequence is calculated. In extraction step 5, the vital parameter can then be extracted from the vital parameter measurement sequence.

FIG. 2 shows a schematic view of a measuring apparatus 6 according to the invention. Measuring apparatus 6 comprises a magnetic induction sensor 7, a first secondary sensor 8 and a second secondary sensor 9 which form a secondary sensor unit 10, an analysis unit 11 and a storage unit 12.

Magnetic induction sensor 7 is connected to analysis unit 11 so as to enable signal transmission, so that the measured induction measurement sequence can be analyzed by analysis unit 11 in combination step 4. Secondary sensor unit 10 has two secondary sensors 8 and 9, which are likewise connected to analysis unit 11 so as to enable signal transmission. Measuring apparatus 6 also comprises storage unit 12, in which the combination function, the induction measurement sequence, the vital parameter measurement sequence and the secondary measurement sequences from secondary sensors 8 and 9 can be stored and can be retrieved by analysis unit 11 at any time.

FIG. 3 shows a schematic view of a possible arrangement of magnetic induction sensor 7 and secondary sensors 8 and 9. Secondary sensor 8 is an optical sensor 13 and is arranged at the center of a coil 14 of magnetic induction sensor 7. Secondary sensor 9 is a capacitive sensor 15 and is arranged alongside magnetic induction sensor 7. An arrangement of this type enables the secondary measurement sequences and the induction measurement sequence to be detected in spatial proximity to one another.

Magnetic induction sensor 7 can detect heart action, for example, and the secondary measurement sequences that contain the influential variable components can be detected by optical sensor 13 and capacitive sensor 15. For example, optical sensor 13 can detect pulse rate and capacitive sensor 15 can detect body movements.

In combination step 4, the influential variable components in the induction measurement sequence and the secondary influential variable components in the secondary measurement sequences can then be offset, and in extraction step 5, a plurality of vital parameters, such as pulse rate, heart action and respiratory movements, for example, can then be extracted with the influencing component offset.

FIG. 4 shows an alternative arrangement of magnetic induction sensor 7 with secondary sensor 8 in the form of optical sensor 13 and with secondary sensor 9 in the form of a self-capacitance sensor 16. In this case, the vital parameters can be detected in a manner similar to the arrangement represented in FIG. 3, and a plurality of vital parameters can be detected simultaneously, free from an influential variable component.

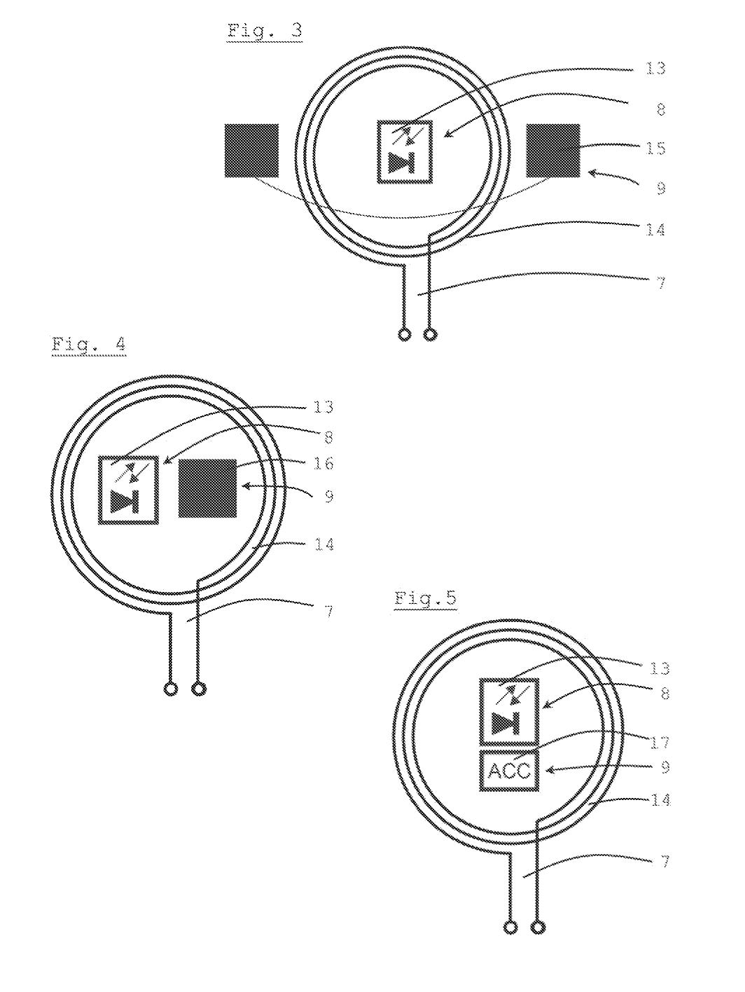

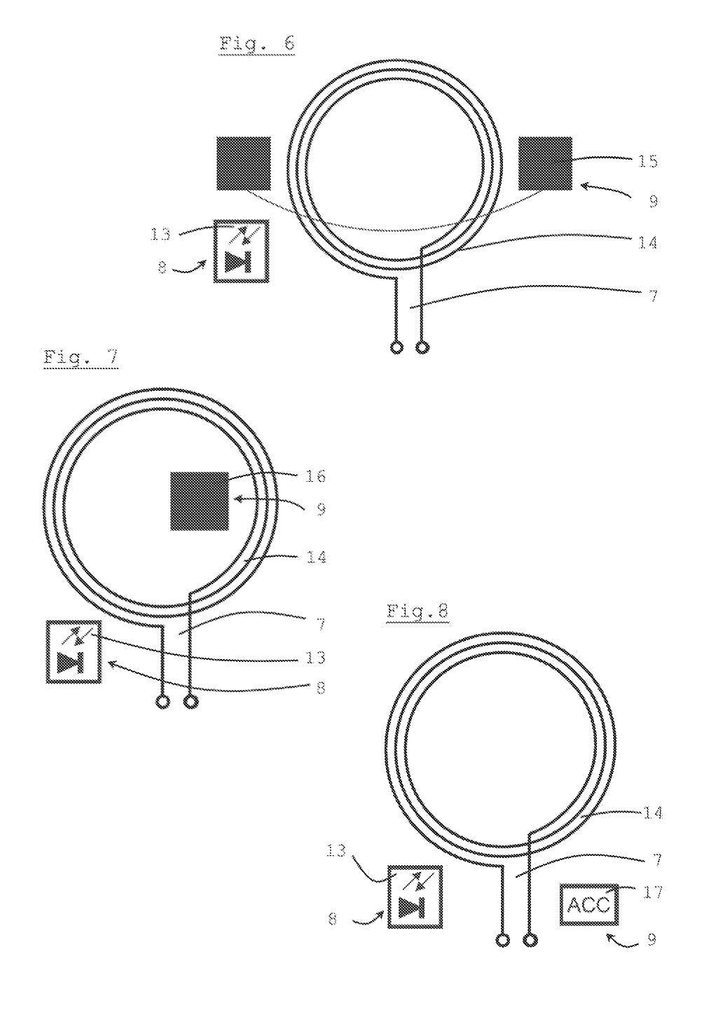

FIG. 5 shows a schematic view of another possible arrangement of magnetic induction sensor 7 and secondary sensors 8 and 9. Secondary sensor 8 is in the form of an optical sensor 13 and secondary sensor 9 is in the form of an acceleration sensor 17.

Magnetic induction sensor 7 detects the induction measurement sequence, and optical sensor 13 and acceleration sensor 17 can obtain the secondary measurement sequences that contain the influential variable components. In combination step 4, both the influential variable components in the induction measurement sequence and secondary influential variable components in the secondary measurement sequences can then be offset.

Subsequent extraction step 5 then enables a plurality of vital parameters to be extracted simultaneously. In this case as well, for example, magnetic induction sensor 7 can detect heart action, the optical sensor can detect pulse rate, and acceleration sensor 17 can detect body movements.

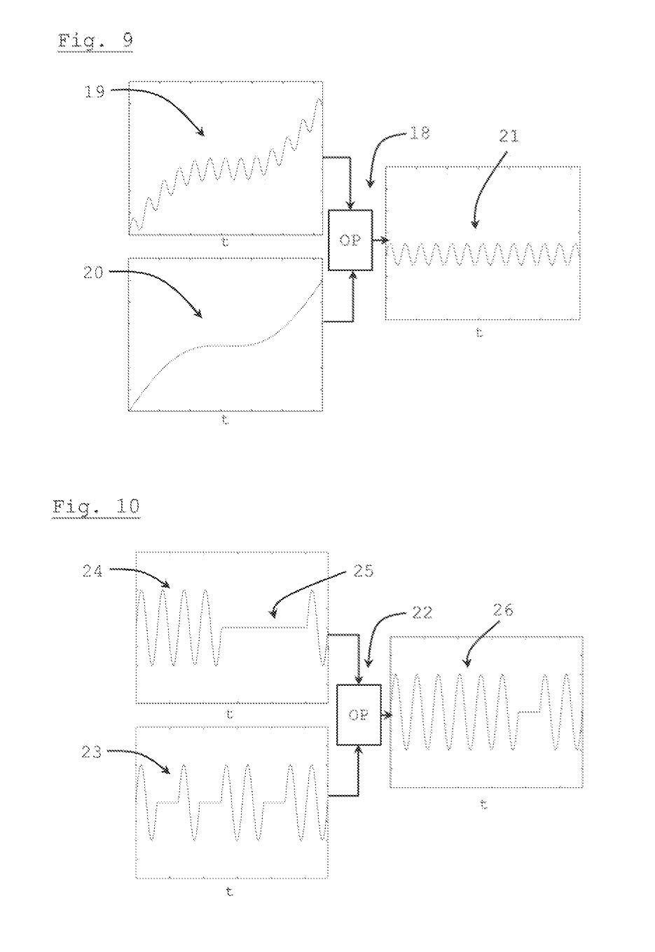

FIG. 6 schematically illustrates an alternative arrangement of magnetic induction sensor 7 and secondary sensors 8 and 9, as shown in FIG. 3. Optical sensor 13 is arranged outside of coil 14 of magnetic induction sensor 7. In the arrangements of magnetic induction sensor 7 and secondary sensors 8 and 9 shown in FIG. 4 and FIG. 5, an alternative arrangement, as shown in FIG. 7 and FIG. 8, is also provided according to the invention. According to the invention, secondary sensors 8 and 9 can both, as shown in FIG. 8, or individually, as shown in FIG. 7, be arranged outside of coil 14 of magnetic induction sensor 7.

The possible arrangements of magnetic induction sensor 7 and secondary sensors 8 and 9 shown in FIGS. 3 to 8 enable the detection of vital parameters at the same spatial measuring point, so that no influential variable components that are based on the measuring position and are different from one another are detected in the induction measurement sequence and in the secondary measurement sequences. As a result, a precise pre-positioning of the individual sensors relative to one another at the measuring point is not necessary, allowing measuring apparatus 6 to be integrated, for example, into automobile seats, hospital beds, examination chairs and articles of clothing, without the detection of vital parameters being negatively influenced by potential undesirable body movements in relation to measuring apparatus 6.

FIG. 9 shows a schematic view of combination step 4 in which a compensation method 18 is applied. In detection step 3, an induction measurement sequence 19 and a secondary measurement sequence 20 are obtained, with the secondary measurement sequence 20 having an influential variable signal sequence that influences induction measurement sequence 19.

Using compensation method 18, the influential variable component in induction measurement sequence 19 is offset, and vital parameter measurement sequence 21 is calculated. In subsequent extraction step 5, the vital parameter can then be extracted from vital parameter measurement sequence 21.

FIG. 10 shows a schematic view of combination step 4 in which a complementary fusion method 22 is applied. In this case, the time dependency of an individual vital parameter in a secondary measurement sequence 23 and in an induction measurement sequence 24 is detected. With complementary fusion method 22, detection errors 25 in induction measurement sequence 24 are then offset, and the time intervals within which the vital parameter is successfully detected are magnified in a vital parameter measurement sequence 26.

FIG. 11 shows a schematic view of combination step 4 in which a source separation method 27 is applied. A first secondary measurement sequence 28, obtained by secondary sensor 8, for example, is dependent on a first time-varying vital parameter, with the first time-varying vital parameter detected from secondary measurement sequence 28 being the influential variable component of an induction measurement sequence 29.

Induction measurement sequence 29 detects a second time-varying vital parameter, which comprises a secondary influential variable component of secondary measurement sequence 28. A second secondary measurement sequence 30 obtained, for example, by secondary sensor 9 detects a further influential variable component of induction measurement sequence 29 and a further secondary influential variable component of secondary measurement sequence 28.

In combination step 4 using source separation method 27, the secondary influential variable component is segregated from secondary measurement sequence 28 based on induction measurement sequence 29 and secondary measurement sequence 30. The influential variable component is also segregated from induction measurement sequence 29 based on secondary measurement sequences 28 and 30. Source separation method 27 can be implemented based on, for example, an independent component analysis, a Kalman filtering or a principal component analysis.

Combination step 4 results in a vital parameter measurement sequence 31 and a vital parameter measurement sequence 32. In subsequent extraction step 5, the two vital parameters can then be extracted from vital parameter measurement sequences 31 and 32. Applying the mutual compensation method enables simultaneous monitoring of a plurality of vital parameters, so that no additional measuring apparatuses are required for detecting additional vital parameters.

1. A measurement method (1) for detecting vital parameters in a human or animal body, in which, in a detection step (3), a magnetic induction sensor (7) detects an induction measurement sequence (19, 24, 29) which is dependent on a time-varying change in at least one vital parameter, characterized in that in the detection step (3), a secondary sensor unit (10) simultaneously obtains a secondary measurement sequence (20, 23, 28, 30), the secondary measurement sequence (20, 23, 28, 30) being dependent on an influential variable signal sequence that influences the induction measurement sequence (19, 24, 29), and in that in a subsequent combination step (4), a predefined combination function is used to calculate at least one vital parameter measurement sequence (21, 26, 31, 32) for a vital parameter detected by the induction measurement sequence (19, 24, 29) from the induction measurement sequence (19, 24, 29) and the secondary measurement sequence (20, 23, 28, 30), thereby improving the accuracy of detection of the vital parameter represented in the vital parameter measurement sequence (21, 26, 31, 32) and the induction measurement sequence (19, 24, 29) by combining the induction measurement sequence (19, 24, 29) with the secondary measurement sequence (20, 23, 28, 30) to form the vital parameter measurement sequence (21, 26, 31, 32).

2. The measurement method (1) according to claim 1, characterized in that the combination function is established in a calibration step (2) prior to the detection step (3).

3. The measurement method (1) according to claim 1, characterized in that in the calibration step (2), parameters of the combination function are established.

4. The measurement method (1) according to claim 1, characterized in that, in an extraction step (5) that follows the combination step (4), the vital parameters are determined from the vital parameter measurement sequence (21, 26, 31, 32).

5. The measurement method (1) according to claim 1, characterized in that the secondary measurement sequence (20, 23, 28, 30) is dependent on at least one additional time-varying vital parameter.

6. The measurement method (1) according to claim 5, characterized in that the time-varying vital parameter detected from the secondary measurement sequence (20, 23, 28, 30) is the influential variable component of the induction measurement sequence (19, 24, 29).

7. The measurement method (1) according to claim 1, characterized in that, in the combination step (4), the induction measurement sequence (19, 24, 29) and the secondary measurement sequence (20, 23, 28, 30) are combined by means of a compensation method (18) to form the vital parameter measurement sequence (21, 26, 31, 32), in order to offset an undesirable influential variable component in the induction measurement sequence (19, 24, 29).

8. The measurement method (1) according to claim 1, characterized in that, in the combination step (4), the induction measurement sequence (19, 24, 29) and the secondary measurement sequence (20, 23, 28, 30) are combined with one another by means of a complementary fusion method (22) to form the vital parameter measurement sequence (21, 26, 31, 32), in order to offset detection errors (25).

9. The measurement method (1) according to claim 5, characterized in that the time-varying vital parameter detected with the induction measurement sequence (19, 24, 29) is a secondary influential variable component of the secondary measurement sequence (20, 23, 28, 30), and in the combination step (4), by means of a mutual compensation method, the secondary influential variable component of the secondary measurement sequence (20, 23, 28, 30) is diminished in the secondary measurement sequence (20, 23, 28, 30) based on the induction measurement sequence (19, 24, 29) and an additional predefined combination function.

10. The measurement method (1) according to claim 1, characterized in that, in the combination step (4), by combining the induction measurement sequence (19, 24, 29) with the secondary measurement sequence (20, 23, 28, 30) by means of a source separation method (27), which is based on a mathematical model which describes a correlation between the vital parameter represented in the induction measurement sequence (19, 24, 29) and the secondary influential variable component represented in the secondary measurement sequence (20, 23, 28, 30) and/or the vital parameter represented in the secondary measurement sequence (20, 23, 28, 30), at least one vital parameter measurement sequence (21, 26, 31, 32) is determined, with a vital parameter detected in the induction measurement sequence (19, 24, 29) and/or in the secondary measurement sequence (20, 23, 28, 30) being represented in the vital parameter measurement sequence (21, 26, 31, 32).

11. The measurement method (1) according to claim 10, characterized in that the source separation method (27) is carried out on the basis of an independent component analysis, a Kalman filtering or a principal component analysis.

12. The measuring apparatus (6) for detecting vital parameters in a human or animal body according to claim 1, characterized in that the measuring apparatus (6) comprises a magnetic induction sensor (7), an analysis unit (11) and a secondary sensor unit (10), the analysis unit (11) being connected to the magnetic induction sensor (7) and the secondary sensor unit (10) so as to enable signal transmission.

13. The measuring apparatus (6) according to claim 12, characterized in that the measuring apparatus (6) comprises a storage unit (12), which is connected to the analysis unit (11) so as to enable signal transmission, and is provided for storing the combination function and/or the induction measurement sequence (19, 24, 29) and/or the secondary measurement sequence (20, 23, 28, 30) and/or the vital parameter measurement sequence (21, 26, 31, 32).

14. The measuring apparatus (6) according to claim 12, characterized in that the secondary sensor unit (10) comprises at least one secondary sensor (8, 9) for detecting the secondary measurement sequence (20, 23, 28, 30).

15. The measuring apparatus (6) according to claim 14, characterized in that the secondary sensor (8, 9) employs a measuring principle different from the principle employed by the magnetic induction sensor (7).

16. The measuring apparatus (6) according to claim 14, characterized in that the secondary sensor (8, 9) is an optical sensor (13).

17. The measuring apparatus (6) according to claim 14, characterized in that the secondary sensor (8, 9) is an acceleration sensor (17).

18. The measuring apparatus (6) according to claim 14, characterized in that the secondary sensor (8, 9) is a sensor based on capacitance coupling.

19. The measuring apparatus (6) according to claim 14, characterized in that the secondary sensor (8, 9) is a distance sensor (15, 16).

20. The measuring apparatus (6) according to claim 1, characterized in that the measuring apparatus (6) is integrated into an automobile seat, an examination chair, a hospital bed or an article of clothing.
